(19) United States
(12) Patent Application Publication
(10) Pub. No.: US 2009/0137914 A1
(43) Pub. Date: May 28, 2009

(54) PORTABLE HYDRAULIC SPHYGMOMANOMETER

(75) Inventor: Shuenn-Tsong Young, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL YANG-MING UNIVERSITY, Taipei City (TW)

(21) Appl. No.: 11/944,937

(22) Filed: Nov. 26, 2007

(51) Int. Cl. A61B 5/022

(52) U.S. Cl. 600/495; 600/490; 600/494

(57) ABSTRACT

The present invention provides a portable hydraulic sphygmomanometer comprising: a manual hydraulic belt, a pressure sensor for detecting the pressure of hydraulic belt, and a compute device for calculating the blood pressure.

### FIELD OF THE INVENTION

The present invention relates to a portable sphygmomanometer.

### BACKGROUND OF THE INVENTION

High blood pressure is a very common condition in modern society. It has been estimated that one in five Americans, around 50 million people, suffer from high blood pressure. In general, more men than women have high blood pressure, and the number of sufferers of both genders increases rapidly with age.

The majority of people with high blood pressure has no symptoms, but if left untreated high blood pressure can lead to major health problems. Consequently the monitoring of blood pressure is vitally important in order to detect cases of high blood pressure and treat them early before health problems can develop.

The conventional sphygmomanometer that functions to measure blood pressure by placing the cuff band on the upper arm has been mainly used. However, the traditional sphygmomanometer is huge and inconvenient. The users cannot measure their blood pressure wherever they need to.

### SUMMARY OF THE INVENTION

The present invention provides a portable hydraulic sphygmomanometer, comprising (a) a hydraulic belt; (b) a pressure sensor operably linked to the hydraulic belt for detecting the pressure of hydraulic belt; and (c) a computer device for calculating the pressure.

The present invention further provides a method for measuring blood pressure, comprising (a) providing a user with the previously mentioned sphygmomanometer and wearing on one hand; (b) pushing a button on a panel by the other hand; (c) releasing the button slowly when hearing the alarm sound; and (d) reading the values of blood pressure on the panel.

### BRIEF DESCRIPTION OF THE DRAWINGS

FIG. 1: the perspective view showing an embodiment of the portable hydraulic sphygmomanometer of the present invention.

FIG. 2: the section view taken along the A-B line in FIG. 1.

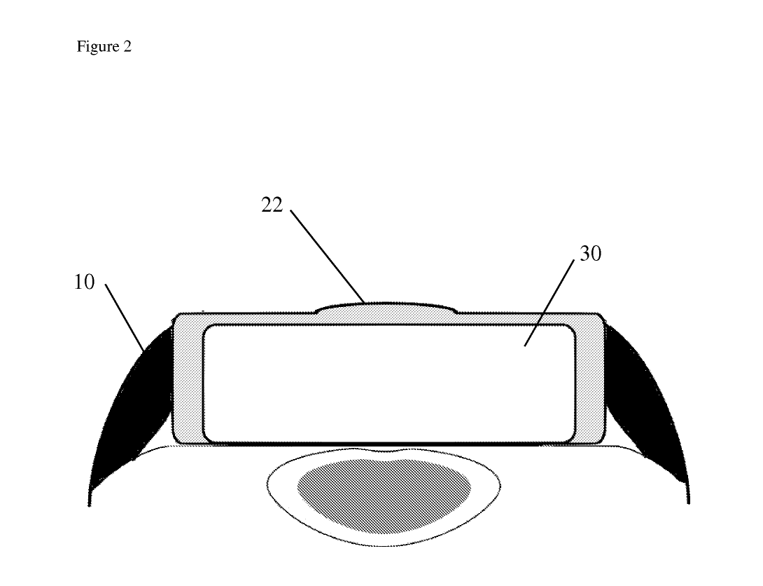

FIG. 3: the section view taken along the □-□ line in FIG. 1.

FIG. 4: the process of decompression in the radial artery which could lead to the calculation of systolic pressure and diastolic pressure.

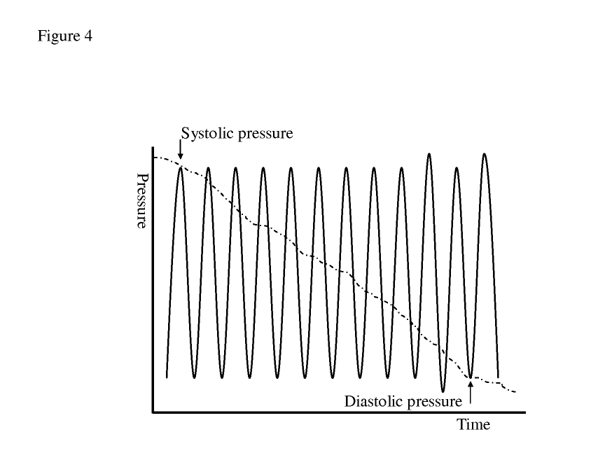

### DESCRIPTION OF MAJOR PARTS IN THE PRESENT INVENTION

10: wrist band
20: main body
21: panel
22: button
30: hydraulic belt
40: pressure sensor
50: computer device
60: alarm device
70: power supply
80: beat detector
90: recorder

### DETAILED DESCRIPTION OF THE INVENTION

Recently, the wrist type sphygmomanometer with the cuff wrapped around the wrist is invented to solve these problems. Although it is much smaller and easier to use than conventional sphygmomanometer, inflating the air bag on the wrist consumes a lot of electricity, and it is also inaesthetic.

The present invention provides a portable hydraulic sphygmomanometer, comprising (a) a hydraulic belt (30); (b) a pressure sensor (40) operably linked to the hydraulic belt (30) for detecting the pressure of hydraulic belt (30); and (c) a computer device (50) for calculating the pressure. The sphygmomanometer further comprises a power supply (70), a panel (21), a button (22) and a wrist band (10) connected to the main body (20) of sphygmomanometer. The panel comprises a button (21) operably linked to the hydraulic belt (30). The force exerted when pushing the button (21) causes the hydraulic belt (30) to compress the blood vessel. Thus, the blood pressure could be calculated during the process of decompression. Also, the panel (21) shows time, date, and values of pressure.

The sphygmomanometer additionally comprises an alarm device (60), a beat detector (80) and a recorder (90). The beat detector (80) detects the disturbance of blood, and the recorder (90) keeps track of the change of pressure in the hydraulic belt (30).

The present invention significantly saves energy by using a hydraulic belt (30) in substitution for a conventional cuff spring. The power supply (70) set forth could be a solar cell, an alkaline battery, a mercury battery or other kinds of batteries. The hydraulic belt (30) is used to detect the blood pressure of brachial or radial arteries. The hydraulic belt (30) further comprises an impermeable package and a liquid. Also the liquid of the hydraulic belt (30) is non-toxic.

The present invention further provides a method for measuring blood pressure, comprising (a) providing a user with the sphygmomanometer of the present invention and wearing on one hand; (b) pushing a button (22) by the other hand; (c) releasing the button (22) slowly when hearing the alarm sound; and (d) reading the values of blood pressure on the panel (21). The button (22) of the sphygmomanometer is operably connected with the hydraulic belt (30). The alarm sound is emitted by the alarm device (60) when the blood stream is blocked.

### EXAMPLE

The present invention was further detailed in the following examples, which were offered by way of illustration and were not intended to limit the invention in any manners. Standard techniques well known in the art or the techniques specifically described below were utilized.

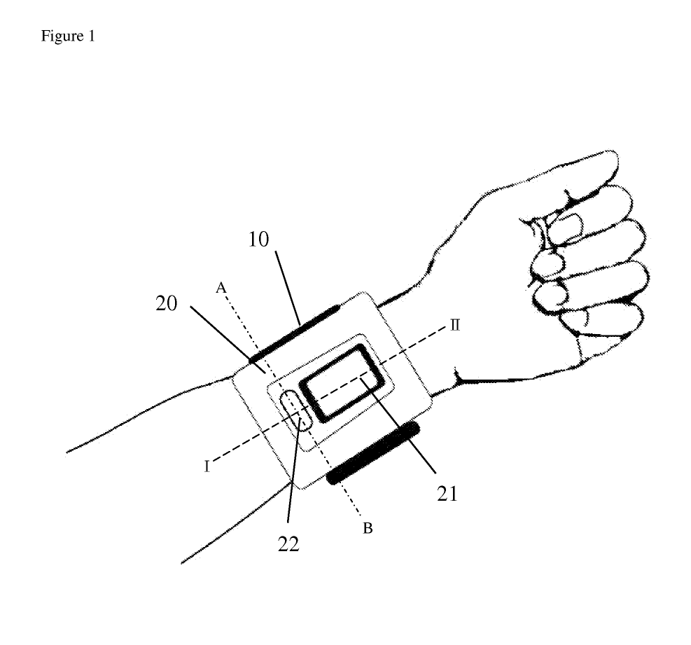

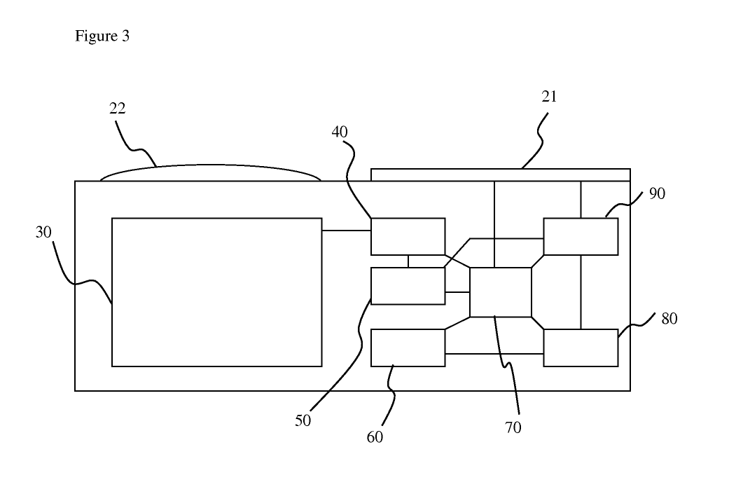

The axiom of the present invention was the same as the prior art. The present invention used a hydraulic belt (30) in substitution for the conventional cuff spring. The user wore the portable hydraulic sphygmomanometer on one hand as FIG. 1 showed. First, the user pressed the button (22) by the other hand until heard the alarm sound was emitted by alarm device (60). In the meanwhile, the blood stream was blocked completely by the hydraulic belt (30). Subsequently, the user released the button (22) slowly and waited for the result showing in the panel (22). The process of decompression in the brachialis was not smooth as the traditional sphygmomanometer (shown in FIG. 3). The first sound of blood disturbance detected by beat detector (80) was systolic pressure, and the last sound of blood disturbance detected by beat detector (80) was diastolic pressure. The wave in the FIG. 3 was arterial pressure, and the oblique line was the process of decompression. However, the electronic apparatus (such as pressure sensor (40), computer device (50) and beat detector (80)), which were accurate and speedy, would overcome this issue. Therefore the present invention provided a novel and more convenient sphygmomanometer to the user.

1. A portable hydraulic sphygmomanometer, comprising
(a) a hydraulic belt;
(b) a pressure sensor operably linked to the hydraulic belt for detecting the pressure of hydraulic belt; and
(c) a computer device for calculating the pressure.

2. The sphygmomanometer of claim 1, wherein the sphygmomanometer further comprising a power supply, a panel, a button and a wrist band connected to the main body of sphygmomanometer.

3. The sphygmomanometer of claim 2, wherein the button operably linked to the hydraulic belt.

4. The sphygmomanometer of claim 2, wherein the panel shows time, date, and values of blood pressure.

5. The sphygmomanometer of claim 1, which further comprises an alarm device, a beat detector and a recorder.

6. The sphygmomanometer of claim 5, wherein the beat detector detects the disturbance of blood.

7. The sphygmomanometer of claim 5, wherein the recorder keeps track of the change of pressure of hydraulic belt according to pressure sensor.

8. The sphygmomanometer of claim 5, wherein the alarm device prompts a user to decompress while the sound of blood disturbance can not be detected by beat detector.

9. The sphygmomanometer of claim 1, wherein the hydraulic belt comprising an impermeable package and a liquid.

10. The sphygmomanometer of claim 9, wherein the liquid is non-toxic.

11. A method for measure the blood pressure, comprising
(a) providing a user with the sphygmomanometer of claim 1 and wearing on one hand;
(b) pushing a button by the other hand;
(c) releasing the button slowly while hearing the alarm sound; and
(d) reading the values of blood pressure on the panel.

12. The method of claim 11, wherein the button is operably connecting with the hydraulic belt.

13. The method of claim 11, wherein the alarm sound is emitted by alarm device when the blood stream is blocked.
